(19) United States
(12) Patent Application Publication
(10) Pub. No.: US 2019/0328936 A1
(43) Pub. Date: Oct. 31, 2019

(54) Polymer Compositions for Stents and Other Implants

(71) Applicants: Mansoor Akhthar Basheerahmed, Little Rock, AR (US); Rakesh Venugopal Duvoor, Louisville, KY (US)

(72) Inventors: Mansoor Akhthar Basheerahmed, Little Rock, AR (US); Rakesh Venugopal Duvoor, Louisville, KY (US)

(21) Appl. No.: 15/963,155

(22) Filed: Apr. 26, 2018

(51) Int. Cl. A61L 31/04; A61L 31/14

(52) U.S. Cl. CPC A61L 31/041; A61L 31/043; A61L 2400/12; A61L 31/148; A61L 2300/254; A61L 31/042

(57) ABSTRACT

Novel formulations for electrospun, electrowoven, and otherwise formed polymer stents and implants are disclosed herein.

### BACKGROUND/FIELD

Stents have gained increasing acceptance in medical practice as mechanical support devices for opening a duct, vessel, or other bodily lumen and ensuring that the lumen remains in an open condition and free of obstructions. In those clinical applications, it is often beneficial to tailor the polymer chemistry of a stent to define specific parameters including for instance resorption rate, elastic modulus, flexural modulus, and impact strength. To that end, novel resorbable stent polymer formulations are provided herein.

### SUMMARY

According to certain embodiments of the present disclosure, a stent for implantation into mammals is composed of polymer blend of a biologically derived protein and a biopolyester.

According to further embodiments of the present disclosure, the stent has relative proportions of 20% PBAT, 40% P4HB, and 40% PHBV by weight.

According to further embodiments of the present disclosure, the stent has relative proportions of 50% PLA, 50% PBS by weight.

According to a further embodiment of the present disclosure, the stent has relative proportions of 50% P3HB, 10% PHBV, 40% Carboxy Methyl Cellulose by weight.

According to a further embodiment of the present disclosure, the stent has relative proportions of 30% P4HB, 20% PHBV, 10% Carboxy Methyl Cellulose, and 30% PCL by weight.

According to a further embodiment of the present disclosure, the stent has relative proportions of 20% PBS, 30% PHBV, 30% soybean polysaccharide, and 20% PTS-co TC by weight.

According to a further embodiment of the present disclosure, the stent has relative proportions of 40% PBS, 20% P3HB, and 40% PLA by weight.

According to a further embodiment of the present disclosure, the stent has relative proportions of 20% PBS, 20% carboxy methyl cellulose, and 60% of one of either (a) Poly (d.l lactide glycolide) or (b) Poly (d,l lactide co-caprolactone).

According to a further embodiment of the present disclosure, the stent has relative proportions of 20% PBAT, 40% soybean polysaccharide, and 40% Bioresorbable PolyUrethane copolymer with PCL.

According to certain embodiment of the present disclosure, a polymer formulation for constructing implants for mammals is provided, the formulation comprising an biologically derived protein and a biopolyester.

According to further embodiments of the present disclosure, the polymer formulation comprises 40% PBS, 50% PCL, and 10% PEG by weight.

According to further embodiments of the present disclosure, the polymer formulation comprises 30% PLA, 20% soybean polysaccharide, 20% PEG, 20% PLGA, and 10% POSS by weight.

According to further embodiments of the present disclosure, the polymer formulation comprises 20% P4HB, 30% PHBV, 30% PPL, 10% PLGA, and 10% POSS by weight.

According to further embodiments of the present disclosure, the polymer formulation comprises 20% PLA, 50% of one of either (a) Poly (d.l lactide glycolide) or (b) Poly (d,l lactide co-caprolactone), and 30% polycitrates.

According to further embodiments of the present disclosure, the polymer formulation comprises 50% PBAT, 30% P3HB, and 20% of one of either (a) Poly (d.l lactide glycolide) or (b) Poly (d,l lactide co-caprolactone).

According to further embodiments of the present disclosure, the polymer formulation comprises 50% PPL, 30% PEG, and 20% of one of either (a) Poly (d.l lactide glycolide) or (b) Poly (d,l lactide co-caprolactone).

### BRIEF DESCRIPTION OF THE FIGURES

FIG. 1 shows an exemplary embodiment of a stent.

Table 1 is a listing of exemplary formulations by weight percentage;

Table 2 is a listing of exemplary formulations from Table 1 by their relative performance characteristics.

### DETAILED DESCRIPTION OF THE FIGURES

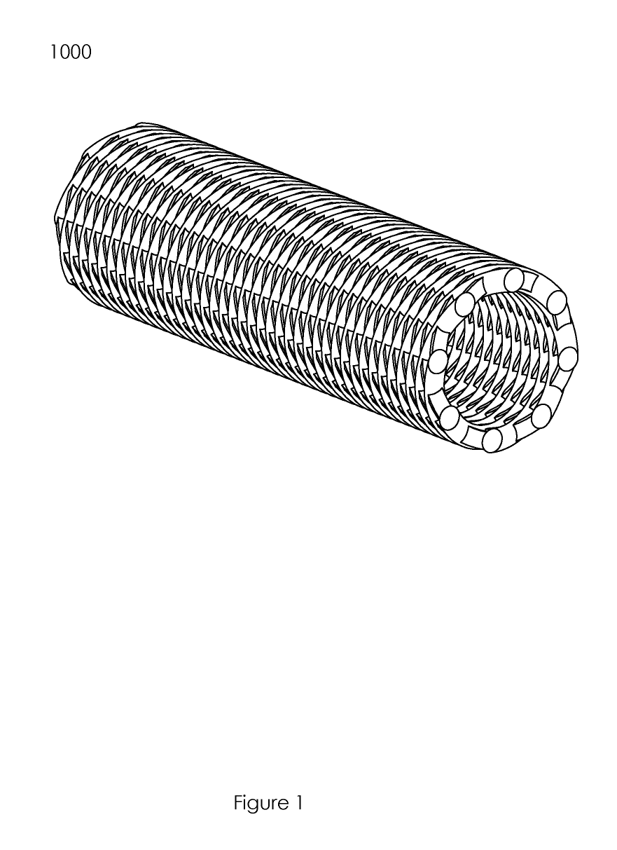

Referring now to FIG. 1, an exemplary embodiment of a stent 1000 is shown, wherein the stent is a generally cylindrical mesh. Further examples of stent geometries are disclosed for instance in U.S. Pat. Nos. 5,217,483 and 5,354,309 which are incorporated by reference in their entirety.

Where the appended formulations are shaped using electrospinning or electroweaving, they should be dissolved in their respective solvents at a range of 20-50 percent molar concentration. Example solvents include but are not limited to DMAc, Toluene, Tatrahydrofuran, Chloroform, DMF, and ester groups.

In some embodiments, the polymer portion of a stent is composed of a single continuous fiber blended from the polymers listed as the formulation. In other embodiments, the stent is composed of a plurality of fibers composed of distinct polymer blends, where the overall mass-percentages of the assembly are approximated by formulations described herein.

Exemplary stents of use in the present invention are generally cylindrically-shaped devices which function to hold open and sometimes expand a segment of a blood vessel or other lumen such as a coronary artery.

Where percentages are provided in the description that follows, these are weight percentages in accordance with accepted practice in the chemical arts. The percentages described below are intended as +/−10% ranges for composition of the resulting plastic part.

### DETAILED DESCRIPTION OF SPECIFIC EMBODIMENTS OF THE PRESENT INVENTION

The present invention comprises formulations which combine percentages of biologically derived proteins with biopolyesters for stents and other implantable structures which have improved mechanical and resorption properties.

Polybutyrate adipate terephthalate, also known as Polyburate, or PBAT is a copolyester of adipic acid, 1, 4-butanediol and dimethylterephthalate. It is a biodegradable polymer used in the present formulations to provide improved overall resilience to the formulations, specifically in that it has a low stiffness and elastic modulus while maintaining a high flexibility and toughness.

Polybutylene succinate, also known as polytetramethylene succinate or PBS is an aliphatic polyester with similar mechanical properties to polypropylene. It is a biodegradable polymer that in the present formulations contributes significantly improved energy absorption.

P3HB or poly-3-hydroxybutyrate is a bio-based polymer related to polyesters. It is biodegradable and contributes a semi-crystalline structure to the formulations below in which it is used.

P4HB or poly-4-hydroxybutyrate is a bio-based polymer related to polyesters. It is biodegradable and lowers the durometer of the resultant structure and increases its elastic modulus.

PHBV or Poly(3-hydroxybutyrate-co-3-hydroxyvalerate is a bio-derived linear aliphatic polyester. It is a biodegradable copolymer, characterized by an amorphous backbone.

PLA or polylactic acid is a naturally derived aliphatic polyester. It is a compostable and bioresorbable polymer which contributes rigidity in the present formulations.

CMC or Carboxymethyl Cellulose is a cellulose derivative with carboxymethyl groups bound to some of the dydroxyl groups of the gluycopryanose monomers that make up the cellulose backbone. It is biodegradable and in the present formulations contributes to faster dissolution and acts as a natural copolymer for the other remaining ingredients.

PCL or Polycaprolactone is a polyester. It is bioresorbable polymer and in the present formulations contributes towards improved rigidity to the stent.

Soybean Polysaccharide is a biodegradable polymer which provides improved tenacity and faster dissolution in the present applications.

PPL or Polypropiolactone is a biodegradable polymer which in the present formulations contributes improved rigidity to the resultant stent or structure.

PEG or polyethylene glycol is a polyether which is a bio-based polymer. In the present formulations, it contributes improved water solubility and rigidity.

PLGA or polylactic-co-glycolic acid is a highly biodegradable and biocompatible copolymer. In the present formulations, it contributes improved rigidity and mechanical performance.

POSS or polyhedral oligomeric silsesquioxane is composed of a hybrid, intermediate (RSiO1.5) between that of silica (SiO2) and silicone (R2SiO). In the present formulation, it contributes high tenacity by means of a nano structure which bridges the functional gap between ceramic and organic materials.

Poly (d.l.lactide co-glycolide) is a biodegradable polymer. Polymer backbone is characterized by atactic and random configuration, constituting lactide and glycolide monomeric units. It's used in drug delivery and medical device applications.

PU-PCL is a Copolymer which consists of both soft elastomeric Polyurethane blocks and hard semicrystalline segments from Polycaprolactone. It is a biobased copolymer.

Polycitrate is a biobased polymer, which contains citric acid. A nanocomposite of polycitrate can establish strong interfacial bonding with inorganic materials and skeletal parts.

### Example Formulations

Where provided below formulations are understood to capture +/−10% of the listed values. All values are listed as weight percentages.

TABLE 1

| | Recipe | | | | | | |
|---|---|---|---|---|---|---|---|
| | | | | | | | Carboxy methyl |
| | | | | P4HB | | | cellulose |
| Ingredient | | PBS | | Soft | PHBV | | Faster |
| role in | PBAT | Energy | P3HB | Structure | Amorphous | PLA | dissolution and |
| STENT | Resiliency | Absorption | Semicrystallinity | and elastic | and elastic | Rigidity | natural polymer |
| 1 | 20 | | | 40 | 40 | | |
| 2 | | 50 | | | | 50 | |
| 3 | | 40 | | | | | |
| 4 | | | | 30 | 20 | | 10 |
| 5 | | | | | | 30 | |
| 6 | | 20 | | | 30 | | |
| 7 | | 40 | 20 | | | 40 | |
| 8 | | | | 20 | 30 | | |

| | Recipe | | | | | | |
|---|---|---|---|---|---|---|---|
| | | Soybean | | | | | POSS |
| | | Polysaccharide | | | PEG | | Nano |
| Ingredient | | Tenacity | | | Water | | material- |
| role in | PCL | and faster | PPL | PTS-co- | soluble | PLGA | high |
| STENT | Rigidity | dissolution | Rigidity | TC | semirigid | Semirigid | tenacity |
| 1 | | | | | | | |
| 2 | | | | | | | |
| 3 | 50 | | | | 10 | | |
| 4 | 30 | | | | | | |
| 5 | | 20 | | | 20 | 20 | 10 |
| 6 | | 30 | | 20 | | | |
| 7 | | | | | | | |
| 8 | | | 30 | | | 10 | 10 |

TABLE 2

| | Recipe | | | | | | | |
|---|---|---|---|---|---|---|---|---|
| | | | | | | | Carboxy methyl | |
| | | | | P4HB | | | cellulose | |
| Ingredient | | PBS | | Soft | PHBV | | Faster | |
| role in | PBAT | Energy | P3HB | Structure | Amorphous | PLA | dissolution and | PCL |
| STENT | Resiliency | Absorption | Semicrystallinity | and elastic | and elastic | Rigidity | natural polymer | Rigidity |
| 9 | | 20 | | | | | 20 | |
| 10 | | | | | | 20 | | |
| 11 | | | 20 | | | | | 40 |
| 12 | | | 50 | | 10 | | 40 | |
| 13 | 20 | | | | | | | |
| 14 | 50 | | | 30 | | | | |
| 15 | | | | | | | | |

| | Recipe | | | | | |
|---|---|---|---|---|---|---|
| | Soybean | | | Poly (d.l | | |
| | Polysaccharide | | PEG | lactide | Bioresorbable | |
| Ingredient | Tenacity | | Water | glycolide) | PU copolymer | |
| role in | and faster | PPL | soluble | Rigid | with PCL | Polycitrates |
| STENT | dissolution | Rigidity | semi-rigid | Polymer | Elasticity | Semirigid |
| 9 | | | | 60 | | |
| 10 | | | | 50 | | 30 |
| 11 | | | | 40 | | |
| 12 | | | | | | |
| 13 | 40 | | | | 40 | |
| 14 | | | | 20 | | |
| 15 | | 50 | 30 | 20 | | |

Relative mechanical characteristics of several of the resultant stents are listed in table 2 below with 9 being greater relative values (of Flexural Modulus, Elasticity, and Impact Strength) and 1 being smaller relative values.

TABLE 2

| | Flexural | Elasticity | Impact |
|---|---|---|---|
| | Modulus | (% elongation | Strength |
| Recipe | (psi) | at break) | (ft-lb/in) |
| 1 | Moderate - 4 | High- 9 | High-9 |
| | (120,000 psi) | (230%) | (2.5 ft-lb/in) |
| 2 | High-9 | Moderate | High-9 |
| 3 | High | Low | Moderate |
| 4 | Moderate | High | Moderate |
| 5 | High | Low | Low |
| 6 | Low | High | Very High |
| 7 | Moderate | Moderate | Moderate |

### Additional Functional Coating:

According to certain further embodiments of the present disclosure, an enzyme which consumes plaques or cholesterol is deposited or electrosprayed onto a polymer stent. Examples of suitable enzymes include serrapeptase and nattokinase. These additional coatings may be applied either as a post-processing step, or as an additional ingredient with the formulations listed above.

According to further still embodiments of the present disclosure, an additional coating including for instance chitosan, carboxy methyl cellulose, sirolumus, or everolimus may be applied in order to minimize the formation of scar tissue around the stent.

According to further still embodiments of the present disclosure, the resultant stent or structure may be coated with an enzyme which consumes plaques or cholesterol such as urokinase or FruA. Similarly, the stent or structure built using the present formulations may be electrosprayed with an anti-inflamitant including for instance serrapeptase and nattokinase.

It is understood that, in light of a reading of the foregoing description, those with ordinary skill in the art will be able to make changes and modifications to the present invention without departing from the spirit or scope of the invention, as defined herein. For example, those skilled in the art may substitute materials supplied by different manufacturers than specified herein without altering the scope of the present invention.

What is claimed is:

1. A stent for implantation into mammals, the stent being composed of polymer blend of an biologically derived protein and a biopolyester.

2. The stent of claim 1, having relative proportions of 20% PBAT, 40% P4HB, and 40% PHBV by weight.

3. The stent of claim 1, having relative proportions of 50% PLA, 50% PBS by weight.

4. The stent of claim 1, having relative proportions of 50% P3HB, 10% PHBV, 40% Carboxy Methyl Cellulose by weight.

5. The stent of claim 1, having relative proportions of 30% P4HB, 20% PHBV, 10% Carboxy Methyl Cellulose, and 30% PCL by weight.

6. The stent of claim 1, having relative proportions of 20% PBS, 30% PHBV, 30% soybean polysaccharide, and 20% PTS-co TC by weight.

7. The stent of claim 1, having relative proportions of 40% PBS, 20% P3HB, and 40% PLA by weight.

8. The stent of claim 1, having relative proportions of 20% PBS, 20% carboxy methyl cellulose, and 60% of one of either (a) Poly (d.l lactide glycolide) or (b) Poly (d,l lactide co-caprolactone).

9. The stent of claim 1, having relative proportions of 20% PBAT, 40% soybean polysaccharide, and 40% Bioresorbable PolyUrethane copolymer with PCL.

10. A polymer formulation for constructing implants for mammals, the formulation comprising an biologically derived protein and a biopolyester.

11. The implantable polymer formulation of claim 10, comprising 40% PBS, 50% PCL, and 10% PEG by weight.

12. The implantable polymer formulation of claim 10, comprising 30% PLA, 20% soybean polysaccharide, 20% PEG, 20% PLGA, and 10% POSS by weight.

13. The implantable polymer formulation of claim 10, comprising 20% P4HB, 30% PHBV, 30% PPL, 10% PLGA, and 10% POSS by weight.

14. The implantable polymer formulation of claim 10, comprising 20% PLA, 50% of one of either (a) Poly (d.l lactide glycolide) or (b) Poly (d,l lactide co-caprolactone), and 30% polycitrates.

15. The implantable polymer formulation of claim 10, comprising 50% PBAT, 30% P3HB, and 20% of one of either (a) Poly (d.l lactide glycolide) or (b) Poly (d,l lactide co-caprolactone).

16. The implantable polymer formulation of claim 10, comprising 50% PPL, 30% PEG, and 20% of one of either (a) Poly (d.l lactide glycolide) or (b) Poly (d,l lactide co-caprolactone).
